(19) United States
(12) Patent Application Publication
(10) Pub. No.: US 2006/0127892 A1
(43) Pub. Date: Jun. 15, 2006

(54) Quantitative diagnostic analysis of hypertonia

(76) Inventors: Andreas Busjahn, Berlin (DE); Friedrich C Luft, Berlin (DE); Florian Lang, Tuebingen (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/472,622

(22) PCT Filed: Mar. 21, 2002
(86) PCT No.: PCT/EP02/03180

§ 371 Date: Apr. 8, 2004

(30) Foreign Application Priority Data
Mar. 21, 2001 (DE) 101 13 876.8

(51) Int. Cl. C12Q 1/68

(52) U.S. Cl. 435/6

(57) ABSTRACT

The invention relates to the application of the direct correlation between the overexpression or the functional molecular modification of human homologs of the sgk family and hypertension for quantitative diagnosis of a particular form of genetically determined hypertension. In particular the invention relates to the detection of a direct link between two different polymorphisms of individual nucleotides in the hsgk1 gene and the genetically determined predisposition to hypertension. The invention further relates to the provision of a diagnostic kit containing antibodies or polynucleotides for detecting the diagnostic targets hsgk1, hsgk2 and hsgk3.

The present invention relates to the direct correlation between the overexpression or the functional molecular modification of human homologs of the sgk family and hypertension. In particular the invention relates to the detection of a direct link between two different polymorphisms of individual nucleotides (single nucleotide polymorphisms=SNP) in the hsgk1 gene and genetically determined predisposition to hypertension.

Numerous extracellular signals lead to intracellular phosphorylation/dephosphorylation cascades, to ensure rapid transmission of these signals from the plasma membrane and its receptors to the cytoplasm and the cell nucleus. The specificity of these reversible signal transduction cascades is made possible by a large number of individual proteins, especially kinases, which transfer a phosphate group onto individual substrates.

Serum- and glucocorticoid-dependent kinase (sgk), a serine/threonine kinase, whose expression is increased by serum and glucocorticoids, was first cloned from rat mammary carcinoma cells (Webster et al., 1993). The human version of sgk, called hsgk1, was cloned from liver cells (Waldegger et al., 1997). It was found that expression of hsgk1 is influenced by regulation of cell volume. To date, no such dependence on cell volume has been detected for the expression of rat sgk. Furthermore, it was found that the rat kinase stimulates the epithelial Na⁺ channel (ENaC) (Chen et al., 1999; Naray-Pejes-Toth et al., 1999). In its turn, the ENaC plays a decisive role in renal Na⁺ excretion. An increased activity of the ENaC leads to increased renal retention of sodium ions, and hence to the development of hypertension.

Finally, two further members of the human sgk gene family were cloned: hsgk2 and hsgk3 (Kobayashi et al., 1999), which are both—as also is hsgk1—activated by insulin and IGF1 via the PI3 kinase pathway. Electrophysiological experiments showed that co-expression of hsgk2 and hsgk3 also leads to a significant increase in activity of the ENaC.

It follows from DE 197 08 173 A1 that hsgk1 possesses a considerable diagnostic potential in many diseases in which changes in cell volume play a decisive pathophysiological role, for example hypernatremia, hyponatremia, diabetes mellitus, renal insufficiency, hypercatabolism, hepatic encephalopathy and microbial or viral infections.

WO 00/62781 had already described activation of the endothelial Na⁺ channel by hsgk1, leading to increase in renal Na⁺ resorption. As this increased renal Na⁺ resorption is associated with hypertension, it was presumed that increased expression of hsgk1 should lead to hypertension, and reduced expression of hsgk1 should eventually lead to hypotension.

A similar relationship between overexpression or hyperactivity of the human homologs hsgk2 and hsgk3 with over-activation of the ENaC, the resulting increased renal Na⁺ resorption and the consequent hypertension was also described in the unpublished, earlier-priority German application with the title “sgk2 and sgk3 as diagnostic and therapeutic targets” (internal designation A 35 048) of 28.08.00. Moreover, the diagnostic potential of the kinases hsgk2 and hsgk3 with respect to arterial hypertension had already been discussed.

The task of the present invention is to find an experimental test for direct correlation, i.e. a direct link between the overexpression or the functional molecular modification of human homologs of the sgk family and hypertension.

A human homolog of the sgk family, which in the above sense includes a functional molecular modification, is to be understood in this context as a homolog of the sgk family that has been mutated in such a way that the properties, especially the catalytic properties or even the substrate specificity of the corresponding protein, are altered.

A further task of the invention is to use this direct correlation or link between the overexpression or the functional molecular modification of human homologs of the sgk family and hypertension in a method for diagnosis of a predisposition to a genetically determined form of hypertension.

Detection of a direct correlation between the overexpression or the functional molecular modification of the human sgk genes and hypertension was provided within the framework of the present invention and in particular was proved experimentally for the example of the hsgk1 gene.

A solution for the above task is therefore the use of this direct correlation between the overexpression or the functional molecular modification of human homologs of the sgk family, especially of the hsgk1 gene, and hypertension, for the diagnosis of a genetically determined form of hypertension.

The above task is achieved in particular in that, within the scope of the present invention, two different SNPs were identified in the hsgk1 gene, which—if they are present in a particular version in the hsgk1 gene—, cause the patient to have a definite tendency to hypertension. The existence of these SNPs in the hsgk1 gene or even in the other human homologs of the sgk gene family can thus be detected in body samples from the patient as a diagnostic indication of a genetically determined predisposition to the development of hypertension.

The above task is further achieved in that a diagnostic method for the quantitative diagnosis of a particular form of genetically determined hypertension is provided, in which the overexpression of a human homolog of the sgk family or the functional molecular modification of these homologs is detected by the quantitative detection of the homologs in the body sample of the patient with antibodies that are directed against the proteins of the homologs, or with polynucleotides, which can hybridize with DNA or mRNA of the homologs under stringent conditions, and by a diagnostic kit that is suitable for carrying out this method.

The kit according to the invention preferably contains the said antibodies that are directed against the hsgk1 protein or the said polynucleotides that can hybridize with the hsgk1 gene under stringent conditions.

This diagnostic kit provides, in particular, antibodies that are specifically directed against regions of the hsgk1 protein that include an hsgk1 protein fragment mutated in the hsgk1 gene corresponding to a specific SNP. The kit can, however, also contain antibodies against the more frequent alleles of the hsgk1 gene or of the other human homologs of the sgk family, with which a modified level of expression of these homologs or of hsgk1 can be detected quantitatively.

Furthermore, the diagnostic kit according to the invention preferably contains polynucleotides that have specific regions which contain one or other version of a hypertension-relevant SNP in the hsgk1 gene and so are suitable for the detection of specific SNPs in the hsgk1 gene of the patient by hybridization under stringent conditions with genomic DNA, cDNA or mRNA from body samples.

The direct correlation according to the invention between hypertension and the human homologs of the sgk family implies that individual mutations could occur in the hsgk1, hsgk2 or hsgk3 genes in some patients, modifying the level of expression or the functional properties of the kinases hsgk1, hsgk2 or hsgk3, and thus leading to a genetically caused tendency to hypertension. Such mutations might occur for example in the regulatory gene regions or in intron sequences of the sgk gene locus and therefore cause overexpression of the corresponding kinase and over-activation of the ENaC. On the other hand, individual differences in the genetic makeup of the sgk locus could also affect the coding region of the gene. Mutations in the coding region could then possibly lead to a functional alteration of the corresponding kinase, e.g. to modified catalytic properties of the kinase. Accordingly, both types of mutations described above could cause increased activation of the ENaC and therefore eventually the formation of a genetically caused form of hypertension in the patient.

These mutations in the human homologs of the sgk family, which give rise to the development of a genetically determined form of hypertension in the patient, are as a rule so-called single nucleotide polymorphisms (SNPs) either in the exon or in the intron region of these homologs. SNPs in the exon region of the hsgk genes can, in their less frequently occurring version—called the mutated version hereinafter—possibly lead to amino acid exchanges in the corresponding hsgk protein and hence to a functional modification of the kinase. SNPs in the intron region or in regulatory sequences of the hsgk genes can, in their mutated version, possibly lead to an altered level of expression of the corresponding kinase.

Within the scope of the present invention, a correlation study was carried out, in which the genotype of the hsgk1 gene of different patients (twins) was compared with their measured systolic and diastolic blood pressure values, which were in each case measured with the body in different positions (sitting, standing, lying down) and evaluated statistically.

Thus it was shown, within the scope of the present invention, that the presence of a (C→T) exchange in exon 8 (1st SNP, see SEQ ID NO. 1) on both alleles (homozygotic TT carriers of the SNPs in exon 8), which does not lead to an amino acid exchange at the protein level (see SEQ ID NO. 2), leads to significantly higher blood pressure values and thus to a genetically determined tendency to hypertension (Table 3).

Furthermore, it was shown that the presence of a (T→C) exchange (2nd SNP), which is localized 551 bp away from the 1st SNP in the donor splicing side in the transition from intron 6 to exon 7, leads in its homozygotic form to lower blood pressure values and thus to a lower genetically determined tendency to hypertension (Table 3).

Since both SNPs in the hsgk1 gene according to the invention do not lead to amino acid exchanges at protein level, the more or less pronounced genetic predisposition to hypertension that is caused by them will probably be based on a modified level of expression of the hsgk1 gene.

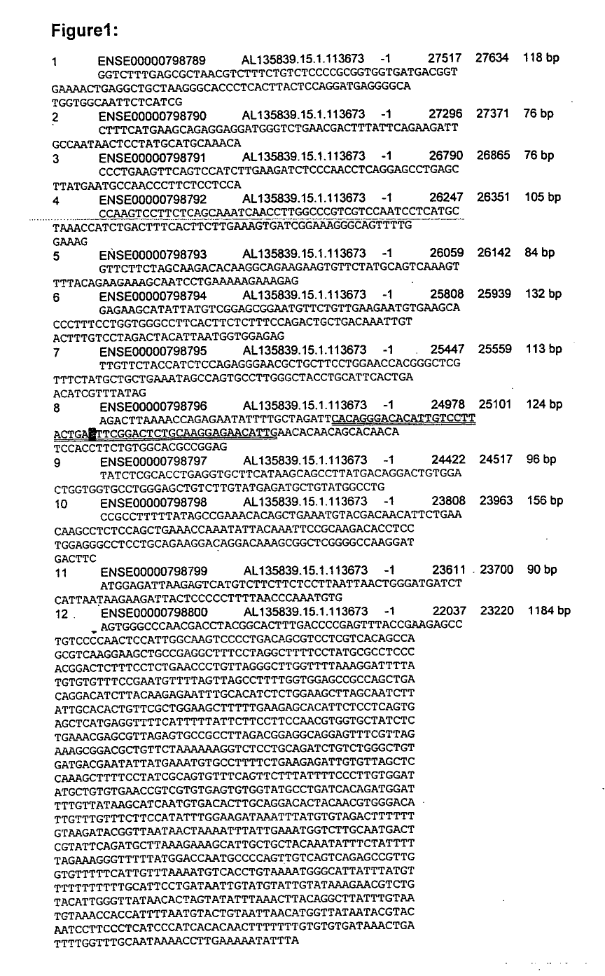

The first SNP in exon 8 (C→T) is explained in more detail in FIG. 1. FIG. 1 shows the individual exons of the hsgk1 gene and described in each case by the exon number, the exon ID, the associated “sequence-contig” and strand, as well as start, end and length of the exon. The exact position of the (C→T) exchange in the framework of the SNPs in exon 8 is indicated by the dark marked C in exon 8. The lighter marking in exon 8 in FIG. 1 indicates the SNP-flanking sequence in the hsgk1 gene, which unambiguously defines the position in the genome.

The second SNP (T→C) in intron 6 was identified by direct sequencing, and is characterized unambiguously in that it is localized in the hsgk1 gene (comprising exons and introns) exactly 551 bp from the first SNP in exon 8 upstream in the donor splicing site of intron 6 to exon 7 of the hsgk1 gene and relates to the exchange of a T for a C.

It could be shown, moreover, that the systolic and diastolic blood pressure values measured with the body in different positions all show a dependence on the genotype of the hsgk1 gene to the same extent (Table 4). Thus, it can be seen from Table 4 that the correlations found between the patients' measured blood pressure and the occurrence of the aforementioned polymorphisms (SNPs) in their hsgk1 genes are in fact statistically significant.

Furthermore, the two SNPs analyzed in the hsgk1 gene show a large imbalance in the frequency of their correlated occurrence (Table 5). Whereas most CC carriers of the SNPs in exon 8 are also TT carriers of the SNPs in intron 6 (64%), the converse is not so (only 2% of the exon 8 TT carriers are also intron 6 CC carriers).

The correlation first detected between the patient's blood pressure and his individual genetic version of the hsgk1 gene locus shows that specific antibodies of polynucleotides, directed against hsgk1, are suitable for the diagnosis of a special, genetically determined tendency to hypertension. This special, genetically caused form of hypertension can be characterized by increased expression of hsgk1, i.e. by overexpression or possibly also by modified functional properties of hsgk1.

Since the two homologous kinases of the sgk family, hsgk2 and hsgk3, also activate the ENaC, according to the invention specific antibodies and polynucleotides that are directed against hsgk2 or hsgk3 are equally suitable for the diagnostic analysis of special genetically determined forms of hypertension.

The finding, according to the invention, that the occurrence of the two SNPs in the hsgk1 gene correlates with a tendency to hypertension, shows that, in particular, polynucleotides that have one or other version of the two SNPs in the hsgk1 gene are especially suitable for the diagnosis of a genetically determined form of hypertension by hybridization with endogenous DNA (cDNA or genomic DNA) or mRNA from a body sample of the patient.

Similarly, according to the present results, antibodies are suitable for the diagnosis of a genetically determined predisposition to hypertension that are directed against specific hypertension-relevant polymorphisms (SNPs) in the hsgk1 protein or one of its human homologs. These SNPs, which also lead to a hypertension-relevant polymorphism at protein level, could in particular be associated with a functional modification of the hsgk1 protein and thus cause a predisposition to hypertension.

The present invention thus relates to the use of the direct correlation, i.e. a direct link between the overexpression or the functional molecular modification of human homologs of the sgk family, especially of hsgk1, and hypertension, for the quantitative diagnosis of a particular form of genetically determined hypertension.

In particular, the two SNPs in the hsgk1 gene that correlate with the tendency to hypertension are used for the quantitative diagnosis of a genetically determined hypertension.

The invention further relates to a method for quantitative diagnosis of a genetically determined form of hypertension, in which the overexpression of a human homolog of the sgk family or the functional molecular modification of these homologs is detected by the quantitative detection of the homologs in the patient's body sample with antibodies that are directed against the proteins of the homologs, or with polynucleotides that can hybridize with genomic DNA, cDNA or mRNA of the homologs under stringent conditions.

In this method of diagnosis according to the invention, the patient's body samples that are used are preferably blood samples or saliva samples, which include cellular material and can be obtained from the patient at relatively little cost. However, other body samples that also include cells, for example tissue samples etc., can also be used. From this cell-containing material of the body samples, either genomic DNA or cDNA or even mRNA can be prepared according to standard methods (Sambrook, J. and Russel, D. W. (2001) Cold Spring Harbor, N.Y., CSHL Press) and if necessary amplified and then hybridized under stringent conditions with polynucleotides that can hybridize specifically with this genomic DNA, cDNA or even mRNA. Furthermore, a protein extract can also be isolated from the cell-containing material of the body samples (blood, saliva, tissue etc.) by standard methods (Sambrook, J. and Russel, D. W. (2001) Cold Spring Harbor, N.Y., CSHL Press), and then the corresponding sgk protein in it can be detected quantitatively by incubation with an antibody that is directed against this protein.

In the method according to the invention, antibodies. against the hsgk1 protein or polynucleotides that can hybridize with genomic DNA, cDNA or mRNA of the hsgk1 gene are preferably used.

In the method according to the invention, in particular polynucleotides are used that can hybridize under stringent conditions with DNA, cDNA or mRNA of a version of the SNP in intron 6 of the hsgk1 gene or a version of the SNP in exon 8 of the hsgk1 gene.

In this context, hybridization under stringent conditions means hybridization under hybridization conditions with respect to hybridization temperature and formamide content of the hybridization solution such as are described in relevant technical literature (Sambrook, J. and Russel, D. W. (2001) Cold Spring Harbor, N.Y., CSHL Press).

In addition the invention relates to a kit for the quantitative diagnosis of a particular form of the genetically determined form of hypertension, containing antibodies that are directed against the human homologs of the sgk protein family, or polynucleotides that can hybridize under stringent conditions with the human homologs of the sgk gene family, or these antibodies and polynucleotides jointly for quantitative determination of the overexpression or the functional molecular modification of these homologs.

The antibodies contained in the kit are preferably directed against the hsgk1 protein, and the polynucleotides contained in the kit can preferably hybridize with the hsgk1gene.

As a special preference, the diagnostic kit can contain polynucleotides that can hybridize with genomic DNA, with cDNA or with mRNA of a version of the SNP in intron 6 (T→C) or of the SNP in exon 8 (C→T).

The present invention is explained in detail by the following examples.

### EXAMPLE 1

Seventy-five pairs of dizygotic twins were recruited for the correlation analysis (Busjahn et al., J. Hypertens. 1996, 14: 1195-1199; Busjahn et al., Hypertension, 1997, 29: 165-170). The test persons all belonged to the German-Caucasian race and came from various regions of Germany. Blood samples were taken from the pairs of twins and from their parents, to verify that they were dizygotic and for further molecular-genetic analyses. Each test person taking part underwent a medical examination beforehand. None of the test persons was known to have a chronic medical condition. After 5 min the test person's blood pressure was measured in the sitting position by a trained doctor using a standardized mercury sphygmomanometer (2 measurements with a time interval of 1 min). The mean value from the two measurements was used as the blood pressure value.

The advantage of dizygotic twins for correlation studies is that they are of exactly the same age and that the external influences on their phenotypes can be regarded as minimal (Martin et al., Nat. Genet., 1997, 17: 387-392).

The importance of studies on twins for elucidating complex genetic diseases was recently described by Martin et al., 1997.

That the pairs of twins were dizygotic was confirmed by amplifying five microsatellite markers using the polymerase chain reaction (PCR). In this analysis of microsatellite markers, fragments of deoxyribonucleic acid (DNA) are amplified by PCR using specific oligonucleotides, which contain highly variable regions. in different human individuals. The high variability in these regions of the genome can be detected by slight differences in size of the amplified fragments, and if there is diversity at the corresponding site of the gene, double bands called microsatellite bands form after separation of the PCR products by gel electrophoresis (Becker et al., J. Reproductive Med. 1997, 42: 260-266).

For the molecular-genetic analysis of the target gene, in this case the hsgk1 gene, three more microsatellite marker regions (d6s472, d6s1038, d6s270) in the immediate vicinity of the hsgk1 locus were amplified by PCR and then compared with the corresponding samples of the other twin and of the parents. In this way it was possible to decide whether the twins had inherited identical or different alleles, relative to the allele under investigation, from their parents. The correlation analysis was carried out using the so-called “structural equation modeling” (SEM) model (Eaves et al., Behav. Genet. 1996, 26: 519-525; Neale, 1997: Mx: Statistical modeling. Box 126 MCV, Richmond, Va. 23298: Department of Psychiatry, 4th edition). This model is based on variance-covariance matrices of the test pairs, which are characterized by the probability that they possess either no, one or two identical alleles. The variance with respect to the phenotype was divided into a variance based on the genetic background of all genes (A), a variance based on the genetic background of the target gene (Q), here the hsgk1 gene, and the variance due to external influences (E).
VAR=A²+Q²+E²

For the three possible allele combinations IBD₀, EBD₁, IBD₂ (IBD=“identical by descent”; 0, 1 or 2 identical alleles), the covariance of a test pair was defined as follows:
COV(IBD₀)=0.5 A² COV(IBD₁)=0.5 A²+0.5 Q² COV(IBD₂)=0.5 A²+Q²

To assess the correlation between the genetic makeup of the hsgk1 locus and the test person's blood pressure, the differences between models that take into account or do not take into account the genetic variance with respect to the hsgk1 target gene were calculated as χ² statistic. For each pair and each gene locus, the allele ratios were calculated by the so-called “multipoint” model (MAPMAKER/SIBS; Kruglyak et al., Am. J. Hum. Genet., 1995, 57: 439-454) based on the parents' genotypes.

The greater informative value of the method of analysis based on variance-covariance estimation, in comparison with the χ² statistic described above (S.A.G.E. Statistical Analysis for Genetic Epidemiology, Release 2.2. Computer program package, Department of Epidemiology and Biostatistics, Case Western Reserve University, Cleveland, Ohio, USA, 1996) was confirmed recently in a simulation study (Fulker et al., Behav. Gen. 1996, 26: 527-532). A level of significance p<0.01 was accepted, in order to ensure a significant correlation with respect to the criteria of Lander and Kruglyak (Lander et al., Nat. Genet., 1995, 11: 241-246).

The results of this correlation study are shown in Table 1.

TABLE 1

| Phenotype | max χ² | p |
|---|---|---|
| systolic blood pressure value (lying) | 4.44 | 0.04 |
| diastolic blood pressure value (lying) | 14.36 | 0.0002 |
| systolic blood pressure value (sitting) | 5.55 | 0.019 |
| diastolic blood pressure value (sitting) | 4.92 | 0.027 |
| systolic blood pressure value (standing) | 1.91 | 0.17 |
| diastolic blood pressure value (standing) | 4.83 | 0.028 |

As can be seen from Table 1, the low values for the levels of significance p, which only exceed the accepted level of significance of p<0.01 slightly, or not at all, prove there is a direct correlation between the genetic variance with respect to the hsgk1 gene site and the phenotypically determined variance of the measured blood pressure.

### EXAMPLE 2

The genomic organization of the hsgk1 gene has already been described (Waldegger et al., Genomics, 51, 299 [1998]), http://www.ensembl.org/Homo_sapiens/geneview?gene=ENSG00000118515).

To identify SNPs whose presence is relevant to predisposition to development of hypertension, firstly the SNPs in the hsgk1 gene published in databanks were investigated as to whether they are true SNPs—and not just sequencing errors—and whether the SNPs are sufficiently polymorphic to provide a basis for diagnostic detection of predisposition to hypertension. The SNP rs 1057293 in exon 8, which relates to exchange of a C for a T, fulfilled the required preconditions (http://www.ensembl.org/Homo_sapiens/snpview?snp=1057293; http://www.ncbi.nlm.nih.gov/SNP/snp_ref.cgi?type=rs&rs=1057293). Furthermore, a second SNP was identified by direct sequencing, which is localized in the hsgk1 gene exactly 551 bp away from the first SNP in the donor splicing site of intron 6 to exon 7 and relates to the exchange of a T for a C. These two SNPs in intron 6 (T→C) and in exon 8 (C→T) were analyzed as described in the following.

After PCR amplification, in each case 1 unit alkaline phosphatase and 1 unit exonuclease I was added, to degenerate the PCR primer and dephosphorylate the dNTPs. PCR was carried out in the following conditions: 95° C. for 10 min, then 35 cycles at 95° for 15 s, followed by 62° C. for 15 s, followed by 72° C. for 30 s, and an extension step at 72° C. for 10 min in a 9600 Thermocycler (Applied Biosystems).

The mini-sequencing reactions were carried out with the primers for intron 6 SNP (T→C) 5′-CTC CTT GCA GAG TCC GAA and for exon 8 SNP (C→T) 5′-ACC AAG TCA TTC TGG GTT GC. 0.15 pmol of purified PCR product was used as template in the sequencing-PCR. For the sequencing-PCR, 25 amplification cycles were carried out with the following individual steps: denaturing 10 s at 96° C., annealing step 10 s at 50° C. and extension step 30 s at 60° C. in a 9600 Thermocycler.

For the same patients whose SNP genotype of the hsgk1 gene was determined, the systolic and diastolic blood pressure values were measured in the lying, standing and sitting position, in order to determine any correlation between SNP genotype of the hsgk1 gene and the blood pressure.

Table 2 shows some demographic twin data and the results of the correlation analysis between the genetic makeup of the hsgk1 gene locus and the measured blood pressure. A strong genetic effect on the measured blood pressure in all positions was demonstrated in the test persons.

TABLE 2

| | monozygotic | dizygotic | | |
|---|---|---|---|---|
| Phenotype | twins | twins | a² (r_{monozyg}/r_{dizyg}) | p (correlation) |
| N | 200 | 132 | | |
| Age y | 29 ± 12 | 31 ± 12 | | |
| Sex (M/F) | 52/148 | 85/47 | | |
| Height (cm) | 169 ± 8 | 170 ± 8 | | |
| Weight (kg) | 65 ± 11 | 67 ± 12 | | |
| Body mass index (BMI) | 22.4 ± 3.5 | 22.8 ± 3.4 | | |
| weight/height² (kg/m²) | | | | |
| Systolic blood pressure | 128 ± 17 | 124 ± 14 | 0.69 | 0.04 |
| (lying) (mmHg) | | | (0.69/0.31) | |
| Diastolic blood pressure | 71 ± 12 | 71 ± 11 | 0.66 | 0.0002 |
| (lying) (mmHg) | | | (0.66/0.42) | |
| Systolic blood pressure | 125 ± 16 | 123 ± 13 | 0.74 | 0.019 |
| (sitting) (mmHg) | | | (0.74/0.38) | |
| Diastolic blood pressure | 73 ± 11 | 73 ± 10 | 0.72 | 0.027 |
| (sitting) (mmHg) | | | (0.72/0.51) | |
| Systolic blood pressure | 124 ± 15 | 122 ± 14 | 0.67 | 0.04 |
| (standing) (mmHg) | | | (0.66/0.48) | |
| Diastolic blood pressure | 80 ± 10 | 79 ± 10 | 0.64 | 0.0002 |
| (standing) (mmHg) | | | (0.63/0.40) | |

Table 3 shows further results of the correlation studies according to the invention. The allele frequencies found for the SNP in exon 8 are C 91% and T 9% and for the SNP in intron 6 they are T 79% and C 21% (the Hardy-Weinberg equilibrium was maintained for both polymorphisms).

The measured blood pressure values showed the same trends in all positions (sitting, lying, standing). Homozygotic CC carriers and heterozygotic CT carriers of the SNP in exon 8 did not show any differences in blood pressure values, but they did show far lower systolic and diastolic blood pressure values than homozygotic TT carriers of the SNP in exon 8.

The corresponding results of the correlation studies are less consistent for the SNP in intron 6 in comparison with the SNP in exon 8. It was found, however, that homozygotic CC carriers of the SNP in intron 6 generally have lower blood pressure values than homozygotic TT carriers and than heterozygotic TC carriers of the SNP in intron 6.

TABLE 3

| | | | | | 2nd | 2nd | 2nd | 2nd |
|---|---|---|---|---|---|---|---|---|
| | 1st SNP | 1st SNP | 1st SNP | 1st SNP | SNP in | SNP in | SNP in | SNP in |
| | in exon 8 | in exon 8 | in exon 8 | in exon 8 | intron 6 | intron 6 | intron 6 | intron 6 |
| Phenotype | CC | CT | TT | CC/CT | TT | CT | CC | TT/CT |
| systolic | 125 ± 15 | 125 ± 18 | 132 ± 14 | 125 ± 16 | 125 ± 16 | 128 ± 18 | 119 ± 6 | 126 ± 16 |
| blood | | | | | | | | |
| pressure | | | | | | | | |
| (lying) | | | | | | | | |
| diastolic | 70 ± 10 | 72 ± 13 | 74 ± 12 | 71 ± 11 | 71 ± 10 | 72 ± 13 | 67 ± 10 | 71 ± 11 |
| blood | | | | | | | | |
| pressure | | | | | | | | |
| (lying) | | | | | | | | |
| systolic | 124 ± 14 | 123 ± 15 | 129 ± 13 | 124 ± 14 | 124 ± 14 | 125 ± 17 | 117 ± 6 | 124 ± 14 |
| blood | | | | | | | | |
| pressure | | | | | | | | |
| (sitting) | | | | | | | | |
| diastolic | 72 ± 10 | 74 ± 10 | 79 ± 9 | 73 ± 10 | 73 ± 10 | 74 ± 11 | 72 ± 9 | 73 ± 10 |
| blood | | | | | | | | |
| pressure | | | | | | | | |
| (sitting) | | | | | | | | |
| systolic | 123 ± 15 | 123 ± 14 | 129 ± 13 | 123 ± 15 | 123 ± 14 | 126 ± 16 | 119 ± 8 | 123 ± 15 |
| blood | | | | | | | | |
| pressure | | | | | | | | |
| (standing) | | | | | | | | |
| diastolic | 79 ± 10 | 81 ± 10 | 84 ± 8 | 80 ± 10 | 80 ± 10 | 82 ± 11 | 78 ± 8 | 80 ± 10 |
| blood | | | | | | | | |
| pressure | | | | | | | | |
| (standing) | | | | | | | | |

Table 4 shows in detail that the genetic makeup of the SNP in intron 6 is substantially equally significant both for the systolic and for the diastolic blood pressure value, regardless of the position in which the blood pressure was measured (sitting, standing, lying). The results for the significance of the genetic makeup of the SNP in exon 8 are similar, but the association of the significance between the measured systolic and diastolic blood pressure values in the different positions is somewhat less pronounced than for the SNP in intron 6.

TABLE 4

| | 2nd SNP | 1st SNP |
|---|---|---|
| Phenotype | in intron 6 | in exon 8 |
| Systolic blood pressure (lying) | <0.01 | <0.05 |
| Diastolic blood pressure (lying) | <0.05 | 0.08 |
| Systolic blood pressure (sitting) | <0.05 | <0.05 |
| Diastolic blood pressure (sitting) | <0.01 | 0.08 |
| Systolic blood pressure (standing) | <0.05 | 0.07 |
| Diastolic blood pressure (standing) | <0.05 | 0.09 |

As can be seen from Table 5, there is a strong correlation equilibrium between the two SNPs analyzed: whereas most CC carriers of the SNP in exon 8 are also TT carriers of the SNP in intron 6 (64%), the reverse is not so (only 2% of the exon 8 TT carriers are also intron 6 CC carriers).

TABLE 5

| | Intron 6 TT | Intron 6 TC | Intron 6 CC |
|---|---|---|---|
| Exon 8 CC | 197 (64%) | 59 (19%) | 3 (1%) |
| Exon 8 CT | 2 (1%) | 30 (10%) | 11 (4%) |
| Exon 8 TT | 0 (0%) | 0 (0%) | 6 (2%) |

1. The use of the direct correlation between the overexpression or the functional molecular modification of human homologs of the sgk family and hypertension for quantitative diagnosis of a particular form of genetically determined hypertension.

2. Use according to claim 1, characterized in that the human homolog of the sgk family is the hsgk1 gene.

3. Use according to claim 2, characterized in that overexpression or functional modification is caused by the nucleotide polymorphism (SNP) in intron 6 (T→C) in the hsgk1 gene.

4. Use according to claim 2, characterized in that overexpression or functional modification is caused by the nucleotide polymorphism (SNP) in exon 8 (C→T) in the hsgk1 gene.

5. A kit for quantitative diagnosis of a particular form of the genetically determined form of hypertension, containing antibodies that are directed against the human homologs of the sgk protein family, or polynucleotides that can hybridize under stringent conditions with the human homologs of the sgk gene family, or these antibodies and polynucleotides jointly for quantitative determination of the overexpression or the functional molecular modification of these homologs.

6. A kit according to claim 5, characterized in that the human homolog of the sgk family is the hsgk1 gene.

7. A kit according to claim 6, characterized in that the antibodies are directed against a version of the hsgk1 protein mutated by an SNP or that the polynucleotides can hybridize under stringent conditions with a version of the hsgk1 gene mutated by an SNP.

8. A kit according to claim 7, characterized in that the polynucleotides can hybridize under stringent conditions with a version of the hsgk1 gene mutated by the SNP in intron 6 (T→C).

9. A kit according to claim 7, characterized in that the polynucleotides can hybridize under stringent conditions with a version of the hsgk1 gene mutated by the SNP in exon 8 (C→T).

10. A method of quantitative diagnosis of a particular form of the genetically determined form of hypertension, in which the overexpression of a human homolog of the sgk family or the functional molecular modification of these homologs is detected by the quantitative detection of the homologs in the patient's body sample with antibodies that are directed against the proteins of the homologs, or with polynucleotides that can hybridize with DNA or mRNA of the homologs under stringent conditions.

11. A method according to claim 10, characterized in that the human homolog of the sgk family is the hsgk1 gene.

12. A method according to claim 10, characterized in that the polynucleotides can hybridize with DNA or mRNA of a version of the SNP in intron 6 (T→C) in the hsgk1 gene under stringent conditions.

13. A method according to claim 10, characterized in that the polynucleotides can hybridize with DNA or mRNA of a version of the SNP in exon 8 (C→T) in the hsgk1 gene under stringent conditions.


SEQUENCE LISTING (16 sequences)

SEQ ID NO: 1 - DNA, 2354 bp, Homo sapiens
  CDS (43)..(1335)
  variation (762): 1st SNP (C to T), silent mutation, i.e. both versions of the SNP result in the amino acid Asp in the amino acid position 240
ggtctttgagcgctaacgtctttctgtctccccgcggtggtgatgacggtgaaaactgaggctgctaagggcaccctcacttactccaggatgaggggcatggtggcaattctcatcgctttcatgaagcagaggaggatgggtctgaacgactttattcagaagattgccaataactcctatgcatgcaaacaccctgaagttcagtccatcttgaagatctcccaacctcaggagcctgagcttatgaatgccaacccttctcctccaccaagtccttctcagcaaatcaaccttggcccgtcgtccaatcctcatgctaaaccatctgactttcacttcttgaaagtgatcggaaagggcagttttggaaaggttcttctagcaagacacaaggcagaagaagtgttctatgcagtcaaagttttacagaagaaagcaatcctgaaaaagaaagaggagaagcatattatgtcggagcggaatgttctgttgaagaatgtgaagcaccctttcctggtgggccttcacttctctttccagactgctgacaaattgtactttgtcctagactacattaatggtggagagttgttctaccatctccagagggaacgctgcttcctggaaccacgggctcgtttctatgctgctgaaatagccagtgccttgggctacctgcattcactgaacatcgtttatagagacttaaaaccagagaatattttgctagattcacagggacacattgtccttactgayttcggactctgcaaggagaacattgaacacaacagcacaacatccaccttctgtggcacgccggagtatctcgcacctgaggtgcttcataagcagccttatgacaggactgtggactggtggtgcctgggagctgtcttgtatgagatgctgtatggcctgccgcctttttatagccgaaacacagctgaaatgtacgacaacattctgaacaagcctctccagctgaaaccaaatattacaaattccgcaagacacctcctggagggcctcctgcagaaggacaggacaaagcggctcggggccaaggatgacttcatggagattaagagtcatgtcttcttctccttaattaactgggatgatctcattaataagaagattactcccccttttaacccaaatgtgagtgggcccaacgacctacggcactttgaccccgagtttaccgaagagcctgtccccaactccattggcaagtcccctgacagcgtcctcgtcacagccagcgtcaaggaagctgccgaggctttcctaggcttttcctatgcgcctcccacggactctttcctctgaaccctgttagggcttggttttaaaggattttatgtgtgtttccgaatgttttagttagccttttggtggagccgccagctgacaggacatcttacaagagaatttgcacatctctggaagcttagcaatcttattgcacactgttcgctggaagctttttgaagagcacattctcctcagtgagctcatgaggttttcatttttattcttccttccaacgtggtgctatctctgaaacgagcgttagagtgccgccttagacggaggcaggagtttcgttagaaagcggacgctgttctaaaaaaggtctcctgcagatctgtctgggctgtgatgacgaatattatgaaatgtgccttttctgaagagattgtgttagctccaaagcttttcctatcgcagtgtttcagttctttattttcccttgtggatatgctgtgtgaaccgtcgtgtgagtgtggtatgcctgatcacagatggattttgttataagcatcaatgtgacacttgcaggacactacaacgtgggacattgtttgtttcttccatatttggaagataaatttatgtgtagacttttttgtaagatacggttaataactaaaatttattgaaatggtcttgcaatgactcgtattcagatgcttaaagaaagcattgctgctacaaatatttctatttttagaaagggtttttatggaccaatgccccagttgtcagtcagagccgttggtgtttttcattgtttaaaatgtcacctgtaaaatgggcattatttatgtttttttttttgcattcctgataattgtatgtattgtataaagaacgtctgtacattgggttataacactagtatatttaaacttacaggcttatttgtaatgtaaaccaccattttaatgtactgtaattaacatggttataatacgtacaatccttccctcatcccatcacacaactttttttgtgtgtgataaactgattttggtttgcaataaaaccttgaaaaatattta

SEQ ID NO: 2 - protein, 431 aa, Homo sapiens
MTVKTEAAKGTLTYSRMRGMVAILIAFMKQRRMGLNDFIQKIANNSYACKHPEVQSILKISQPQEPELMNANPSPPPSPSQQINLGPSSNPHAKPSDFHFLKVIGKGSFGKVLLARHKAEEVFYAVKVLQKKAILKKKEEKHIMSERNVLLKNVKHPFLVGLHFSFQTADKLYFVLDYINGGELFYHLQRERCFLEPRARFYAAEIASALGYLHSLNIVYRDLKPENILLDSQGHIVLTDFGLCKENIEHNSTTSTFCGTPEYLAPEVLHKQPYDRTVDWWCLGAVLYEMLYGLPPFYSRNTAEMYDNILNKPLQLKPNITNSARHLLEGLLQKDRTKRLGAKDDFMEIKSHVFFSLINWDDLINKKITPPFNPNVSGPNDLRHFDPEFTEEPVPNSIGKSPDSVLVTASVKEAAEAFLGFSYAPPTDSFL

SEQ ID NO: 3 - DNA, 118 bp, Homo sapiens
ggtctttgagcgctaacgtctttctgtctccccgcggtggtgatgacggtgaaaactgaggctgctaagggcaccctcacttactccaggatgaggggcatggtggcaattctcatcg

SEQ ID NO: 4 - DNA, 76 bp, Homo sapiens
ctttcatgaagcagaggaggatgggtctgaacgactttattcagaagattgccaataactcctatgcatgcaaaca

SEQ ID NO: 5 - DNA, 76 bp, Homo sapiens
ccctgaagttcagtccatcttgaagatctcccaacctcaggagcctgagcttatgaatgccaacccttctcctcca

SEQ ID NO: 6 - DNA, 105 bp, Homo sapiens
ccaagtccttctcagcaaatcaaccttggcccgtcgtccaatcctcatgctaaaccatctgactttcacttcttgaaagtgatcggaaagggcagttttggaaag

SEQ ID NO: 7 - DNA, 84 bp, Homo sapiens
gttcttctagcaagacacaaggcagaagaagtgttctatgcagtcaaagttttacagaagaaagcaatcctgaaaaagaaagag

SEQ ID NO: 8 - DNA, 132 bp, Homo sapiens
gagaagcatattatgtcggagcggaatgttctgttgaagaatgtgaagcaccctttcctggtgggccttcacttctctttccagactgctgacaaattgtactttgtcctagactacattaatggtggagag

SEQ ID NO: 9 - DNA, 113 bp, Homo sapiens
ttgttctaccatctccagagggaacgctgcttcctggaaccacgggctcgtttctatgctgctgaaatagccagtgccttgggctacctgcattcactgaacatcgtttatag

SEQ ID NO: 10 - DNA, 124 bp, Homo sapiens
  variation (58): 1st SNP (C to T), silent mutation
agacttaaaaccagagaatattttgctagattcacagggacacattgtccttactgacttcggactctgcaaggagaacattgaacacaacagcacaacatccaccttctgtggcacgccggag

SEQ ID NO: 11 - DNA, 96 bp, Homo sapiens
tatctcgcacctgaggtgcttcataagcagccttatgacaggactgtggactggtggtgcctgggagctgtcttgtatgagatgctgtatggcctg

SEQ ID NO: 12 - DNA, 156 bp, Homo sapiens
ccgcctttttatagccgaaacacagctgaaatgtacgacaacattctgaacaagcctctccagctgaaaccaaatattacaaattccgcaagacacctcctggagggcctcctgcagaaggacaggacaaagcggctcggggccaaggatgacttc

SEQ ID NO: 13 - DNA, 90 bp, Homo sapiens
atggagattaagagtcatgtcttcttctccttaattaactgggatgatctcattaataagaagattactcccccttttaacccaaatgtg

SEQ ID NO: 14 - DNA, 1184 bp, Homo sapiens
agtgggcccaacgacctacggcactttgaccccgagtttaccgaagagcctgtccccaactccattggcaagtcccctgacagcgtcctcgtcacagccagcgtcaaggaagctgccgaggctttcctaggcttttcctatgcgcctcccacggactctttcctctgaaccctgttagggcttggttttaaaggattttatgtgtgtttccgaatgttttagttagccttttggtggagccgccagctgacaggacatcttacaagagaatttgcacatctctggaagcttagcaatcttattgcacactgttcgctggaagctttttgaagagcacattctcctcagtgagctcatgaggttttcatttttattcttccttccaacgtggtgctatctctgaaacgagcgttagagtgccgccttagacggaggcaggagtttcgttagaaagcggacgctgttctaaaaaaggtctcctgcagatctgtctgggctgtgatgacgaatattatgaaatgtgccttttctgaagagattgtgttagctccaaagcttttcctatcgcagtgtttcagttctttattttcccttgtggatatgctgtgtgaaccgtcgtgtgagtgtggtatgcctgatcacagatggattttgttataagcatcaatgtgacacttgcaggacactacaacgtgggacattgtttgtttcttccatatttggaagataaatttatgtgtagacttttttgtaagatacggttaataactaaaatttattgaaatggtcttgcaatgactcgtattcagatgcttaaagaaagcattgctgctacaaatatttctatttttagaaagggtttttatggaccaatgccccagttgtcagtcagagccgttggtgtttttcattgtttaaaatgtcacctgtaaaatgggcattatttatgtttttttttttgcattcctgataattgtatgtattgtataaagaacgtctgtacattgggttataacactagtatatttaaacttacaggcttatttgtaatgtaaaccaccattttaatgtactgtaattaacatggttataatacgtacaatccttccctcatcccatcacacaactttttttgtgtgtgataaactgattttggtttgcaataaaaccttgaaaaatattta

SEQ ID NO: 15 - DNA, 18 bp, Artificial Sequence
  Description of Artificial Sequence Synthetic primer
ctccttgcagagtccgaa

SEQ ID NO: 16 - DNA, 20 bp, Artificial Sequence
  Description of Artificial Sequence Synthetic primer
accaagtcattctgggttgc